(19) United States
(12) Patent Application Publication
(10) Pub. No.: US 2023/0149510 A1
(43) Pub. Date: May 18, 2023

(54) METHOD FOR ALLEVIATING ARTHRITIS USING EPIDERMAL GROWTH FACTOR

(71) Applicant: Lovin BioMed Co., Ltd., Kaohsiung City (TW)

(72) Inventors: Le-Shin Chang, Kaohsiung City (TW); Yen-Shuo Chiu, New Taipei City (TW); Chi-Chien Lin, Taichung City (TW); Shih-Chao Lin, Keelung City (TW); Shang-Chen Chia, Kaohsiung City (TW)

(21) Appl. No.: 17/853,035

(22) Filed: Jun. 29, 2022

(30) Foreign Application Priority Data
Nov. 17, 2021 (TW) 110142734

(51) Int. Cl. A61K 38/18; A61K 31/737; A61K 31/728; A61P 19/02; A23L 33/125

(52) U.S. Cl. CPC A61K 38/1808; A61K 31/737; A61K 31/728; A61P 19/02; A23L 33/125; A23V 2002/00

(57) ABSTRACT

Disclosed herein is a method for alleviating arthritis, which includes administering to a subject in need thereof a composition containing epidermal growth factor.

### CROSS-REFERENCE TO RELATED APPLICATION

This application claims priority of Taiwanese Invention Patent Application No. 110142734, filed on Nov. 17, 2021.

### FIELD

The present disclosure relates to a method for alleviating arthritis using epidermal growth factor (EGF), and more particularly to a method for alleviating arthritis using a composition containing EGF, a glycosaminoglycan, and hyaluronic acid.

### BACKGROUND

Arthritis is a degenerative disease that occurs in various joints (such as hands, feet, wrists, elbows, knees, hips, and ankles). Common types of arthritis include degenerative arthritis (also known as osteoarthritis (OA)), rheumatoid arthritis (RA), and gouty arthritis (GA). Arthritis might cause joint inflammation, swelling, deformity, pain, atrophy, stiffness, and eventually impaired mobility.

The current clinical treatment strategies for arthritis are use of steroids to reduce inflammation and use of hyaluronic acid (HA) to provide lubrication, along with physical rehabilitation, so as to control cartilage damage. However, these treatment strategies cannot achieve the desired effect, and in severe cases, artificial joint replacement by surgery is required.

Platelet-rich plasma (PRP) is a concentrated solution obtained by removing red blood cells from whole blood, and contains a large number of platelets and a variety of growth factors, among which the content of insulin-like growth factor I (IGF-I) is the highest, the content of transforming growth factor-β (TGF-β) is the second, while the content of epidermal growth factor (EGF) is extremely low (about 1/450 times the content of IGF-I) (Ha C. W. et al. (2019), Arthroscopy, 35:2878-2884).

It has been reported in Gazendam A. et al. (2021), Br. J. Sports Med., 55:256-261 that there was no statistical difference between PRP injections and placebo (i.e., saline) injections in improving joint function or pain. In particular, the therapeutic efficacy achieved with the combination of PRP and hyaluronic acid was lower than that achieved with PRP alone. In addition, it has been reported in Nah S. S. et al. (2010), Rheumatol. Int., 30:443-449 that EGF is abundantly expressed in patients with RA, and is therefore thought to be involved in the pathogenesis of arthritis, especially the inflammatory process of RA.

Moreover, it has been reported in Swanson C. D. et al. (2012), J. Immunol., 188:3513-3521 that epidermal growth factor receptor (EGFR) is abundantly expressed in mice with collagen-induced arthritis and in patients with RA, and is therefore thought to be involved in the pathogenesis of arthritis. Swanson C. D. et al. also disclosed that an EGFR inhibitor (i.e., erlotinib) can alleviate collagen-induced arthritis, so EGFR is expected to be used as a therapeutic target.

It has been reported in Sun H. et al. (2018), EBioMedicine, 32:223-233 that EGFR is highly activated in patients with OA, resulting in joint damage. Sun H. et al. also disclosed that another EGFR inhibitor (i.e., gefitinib) can improve symptoms in mice with OA, and is therefore expected to be an effective therapeutic agent.

In spite of the aforesaid, there is still a need to develop an effective way for alleviating arthritis.

### SUMMARY

Accordingly, an object of the present disclosure is to provide a method for alleviating arthritis, which can alleviate at least one of the drawbacks of the prior art, and which includes administering to a subject in need thereof a composition containing epidermal growth factor.

### BRIEF DESCRIPTION OF THE DRAWINGS

Other features and advantages of the present disclosure will become apparent in the following detailed description of the embodiments with reference to the accompanying drawings, of which:

FIG. 1 shows the diameter of the right knee joint determined in each group of Example 1, infra, in which the symbols “*”, “**”, and “***” respectively represent p<0.05, p<0.01, and p<0.001 (compared with the pathological control group);

FIG. 2 shows the scale of osteoarthritis (OA) determined in each group of Example 1, infra, in which the symbols “*” and “***” respectively represent p<0.05 and p<0.001 (compared with the pathological control group);

FIG. 3 shows the content of cartilage oligomeric matrix protein (COMP) determined in each group of Example 1, infra, in which the symbol “**” represents p<0.01 (compared with the pathological control group);

FIG. 4 shows the contents of TNF-α, IL-1β, IL-6, and IL-17A determined in each group of Example 1, infra, in which the symbols “*”, “**”, and “***” respectively represent p<0.05, p<0.01, and p<0.001 (compared with the pathological control group);

FIG. 5 shows the contents of PGE2 and NO determined in each group of Example 1, infra, in which the symbols “*”, “**”, and “***” respectively represent p<0.05, p<0.01, and p<0.001 (compared with the pathological control group);

FIG. 6 shows the scale of rheumatoid arthritis (RA) determined in each group of Example 2, infra;

FIG. 7 shows the scales of inflammatory cell infiltration, synovial hyperplasia, and cartilage surface erosion determined in each group of Example 2, infra, in which the symbols “*” and “***” respectively represent p<0.05 and p<0.001 (compared with the pathological control group);

FIG. 8 shows the TNF-α, IL-1β, and IL-6 contents determined in each group of Example 2, infra, in which the symbols “*”, “**”, and “***” respectively represent p<0.05, p<0.01, and p<0.001 (compared with the pathological control group); and

FIG. 9 shows the IL-17A, IFN-γ, and IL-8 contents determined in each group of Example 2, infra, in which the symbols “*”, “**”, and “***” respectively represent p<0.05, p<0.01, and p<0.001 (compared with the pathological control group).

### DETAILED DESCRIPTION

For the purpose of this specification, it will be clearly understood that the word “comprising” means “including but not limited to”, and that the word “comprises” has a corresponding meaning.

It is to be understood that, if any prior art publication is referred to herein, such reference does not constitute an admission that the publication forms a part of the common general knowledge in the art, in Taiwan or any other country.

Unless defined otherwise, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a method for alleviating arthritis, which includes administering to a subject in need thereof a composition containing epidermal growth factor (EGF).

As used herein, the term “alleviating” or “alleviation” refers to at least partially reducing, ameliorating, relieving, controlling, treating or eliminating one or more clinical signs of a disease or disorder; and lowering, delaying, stopping or reversing the progression of severity regarding the condition or symptom being treated and preventing or decreasing the likelihood or probability thereof.

As used herein, the term “administration” or “administering” means introducing, providing or delivering a pre-determined active ingredient to a subject by any suitable routes to perform its intended function.

As used herein, the term “subject” refers to any animal of interest, such as humans, monkeys, cows, sheep, horses, pigs, goats, dogs, cats, mice, rats, rabbits, elephants, bears, and deer.

In certain embodiments, the subject is a human.

According to the present disclosure, the arthritis may be selected from the group consisting of osteoarthritis (OA), rheumatoid arthritis (RA), gouty arthritis (GA), psoriatic arthritis (PA), infectious arthritis (IA), and combinations thereof. In an exemplary embodiment, the arthritis is osteoarthritis. In another exemplary embodiment, the arthritis is rheumatoid arthritis.

According to the present disclosure, the epidermal growth factor suitable for use in this disclosure may be derived from humans or other animals, plants, and microorganisms, and may be obtained as commercial products, or may be prepared using techniques well-known to those skilled in the art. For example, the epidermal growth factor may be a natural product isolated from a biological material (such as human tissues), or a recombinant protein or a functional fragment thereof obtained by genetic engineering. In certain embodiments, the epidermal growth factor is a recombinant human epidermal growth factor (rhEGF).

According to the present disclosure, the composition may further contain a glycosaminoglycan and hyaluronic acid (HA).

In certain embodiments, the glycosaminoglycan may be selected from the group consisting of chondroitin sulfate, keratan sulfate, heparan sulfate, glucosamine sulfate, and combinations thereof. In an exemplary embodiment, the glycosaminoglycan is chondroitin sulfate.

In certain embodiments, the epidermal growth factor, the glycosaminoglycan, and the hyaluronic acid in the composition are present in a weight ratio ranging from 1:10:100 to 1:1000:5000. In an exemplary embodiment, the epidermal growth factor, the glycosaminoglycan, and the hyaluronic acid are present in a weight ratio of 1:100:1000.

According to the present disclosure, the composition is substantially free of platelet-rich plasma (PRP).

According to the present disclosure, the composition may be formulated as a food product using a standard technique well known to one of ordinary skill in the art. For example, the composition may be directly added to an edible material or may be used to prepare an intermediate composition (e.g., a food additive or a premix) suitable to be subsequently added to the edible material.

As used herein, the term “food product” refers to any article or substance that can be ingested by a subject into the body thereof. Examples of the food product may include, but are not limited to, milk powders, fermented milk, yogurt, butter, beverages (e.g., tea, coffee, etc.), functional beverages, a flour product, baked foods, confectionery, candies, fermented foods, animal feeds, health foods, infant foods, and dietary supplements.

According to the present disclosure, the composition may be prepared in the form of a pharmaceutical composition. The pharmaceutical composition may be formulated into a dosage form suitable for oral administration, parenteral administration, or topical administration using technology well known to those skilled in the art.

For parenteral administration, the pharmaceutical composition according to the present disclosure may be formulated into an injection, e.g., a sterile aqueous solution or a dispersion.

The pharmaceutical composition according to the present disclosure may be administered via one of the following parenteral routes: intraperitoneal injection, intrapleural injection, intramuscular injection, intravenous injection, intraarterial injection, intraarticular injection, intrasynovial injection, intrathecal injection, intracranial injection, intraepidermal injection, subcutaneous injection, intradermal injection, intralesional injection, implant administration, and sublingual administration. In certain embodiments, the pharmaceutical composition may be administered via intraperitoneal injection or intraarticular injection.

According to the present disclosure, the dosage form suitable for oral administration includes, but is not limited to, sterile powders, tablets, troches, lozenges, pellets, capsules, dispersible powders or granules, solutions, suspensions, emulsions, syrup, elixir, slurry, and the like.

According to the present disclosure, the pharmaceutical composition may be formulated into an external preparation suitable for topical application to the skin using technology well known to those skilled in the art. The external preparation includes, but is not limited to, emulsions, gels, ointments, creams, patches, liniments, powder, aerosols, sprays, lotions, serums, pastes, foams, drops, suspensions, salves, scaffolds, and bandages.

According to the present disclosure, the pharmaceutical composition may further include a pharmaceutically acceptable carrier widely employed in the art of drug-manufacturing. For instance, the pharmaceutically acceptable carrier may include one or more of the following agents: solvents, buffers, emulsifiers, suspending agents, decomposers, disintegrating agents, dispersing agents, binding agents, excipients, stabilizing agents, chelating agents, diluents, gelling agents, preservatives, wetting agents, lubricants, absorption delaying agents, liposomes, scaffolds, and the like. The choice and amount of the aforesaid agents are within the expertise and routine skills of those skilled in the art.

The dose and frequency of administration of the pharmaceutical composition may vary depending on the following factors: the severity of the illness or disorder to be treated, routes of administration, and age, physical condition and response of the subject to be treated. In general, the pharmaceutical composition may be administered in a single dose or in several doses.

The disclosure will be further described by way of the following examples. However, it should be understood that the following examples are solely intended for the purpose of illustration and should not be construed as limiting the disclosure in practice.

### EXAMPLES

General Experimental Materials:

A. Epidermal growth factor (EGF) (Cat. No. 02-108), chondroitin sulfate (CS) (Cat. No. 05-103), and hyaluronic acid (HA) (Cat. No. 07-113) used in the following experiments were purchased from Joycom Bio-chem Co., Ltd., Taiwan.

General Procedures:

1. Statistical Analysis

All the experiments described below were performed in triplicates. The experimental data are expressed as mean±standard error of the mean (SEM). Statistical analysis was conducted using GraphPad Prism 8.4 (GraphPad Software, San Diego, USA). All of the experimental data were analyzed using one-way analysis of variance (ANOVA) followed by Tukey's test, so as to evaluate the differences between the groups. Statistical significance is indicated by p<0.05.

### Example 1. Evaluation for the Effect of EGF in Alleviating Osteoarthritis (OA)

A. Preparation of mixture containing EGF, CS, and HA EGF, CS, and HA were mixed in a weight ratio of 1:100:1000 in sterile water, so as to obtain a mixture containing EGF (20 μg/g), CS, and HA.

B. Experimental Rats

Male Sprague Dawley rats (6 weeks old, with a body weight of approximately 200 to 300 g) used in the following experiments were purchased from National Laboratory Animal Center, Taipei City, Taiwan. All the experimental mice were housed in an animal room under the following laboratory conditions: an alternating 12-hour light and 12-hour dark cycle, a temperature maintained at 21° C. to 24° C., and a relative humidity maintained at 45% to 70%. Furthermore, water and feed were provided ad libitum for all the experimental mice. All experimental procedures involving the experimental mice were in compliance with the legal provision of the Institutional Animal Care and Use Committee of National Chung Hsing University, Taiwan, and were carried out according to the Guide for the Care and Use of Laboratory Animals of National Institutes of Health (NIH).

C. Induction of OA and Administration of EGF

The Sprague Dawley rats were divided into five groups, including one normal control group, one pathological control group, one comparative group, and two experimental groups (i.e., experimental groups 1 to 2) (n=5 rats in each group).

A respective one of EGF and the mixture containing EGF, CS, and HA was dissolved in phosphate-buffered saline (PBS), so as to obtain an EGF solution and a mixture solution.

The right knee joint of the respective rat in the pathological control group, the comparative group, and the experimental groups 1 and 2 was intraarticularly injected with 30 μL of a monosodium iodoacetate (MIA) solution (50 g/L, in 0.9% normal saline). In addition, the right knee joint of the respective rat in the normal control group was intraarticularly injected with 30 μL of 0.9% normal saline.

On day 3 and day 14 after injection of the MIA solution, the right knee joint of the respective rat in the comparative group was intraarticularly injected with 30 μL of 1% HA, the right knee joint of the respective rat in the experimental group 1 was intraarticularly injected with 30 μL of the EGF solution (containing 20 μg/g EGF), the right knee joint of the respective rat in the experimental group 2 was intraarticularly injected with 30 μL of the mixture solution (containing 20 μg/g EGF), and the right knee joint of the respective rat in the pathological control group was intraarticularly injected with 30 μL of 0.9% normal saline. In addition, the rats of the normal control group received no treatment.

D. Determination of Diameter of Right Knee Joint

At the end of day 28 after injection of the MIA solution, the right knee joint of the respective rat in each group was subjected to determination of diameter using a caliper.

The data thus obtained were analyzed according to the method described in section 1 of “General Procedures”.

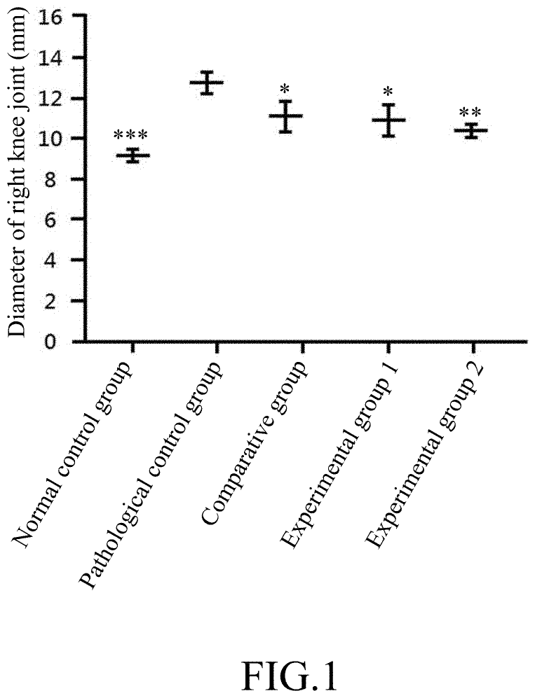

Referring to FIG. 1, the diameters of the right knee joints determined in the comparative group and the experimental groups 1 and 2 were each lower than that determined in the pathological control group. In particular, the diameter of the right knee joint determined in the experimental group 2 was similar to that determined in the normal control group.

These results indicate that EGF can exhibit satisfactory efficacy in alleviating OA, and this efficacy can be enhanced when EGF is used in combination with CS and HA.

E. Preparation of Biological Samples

After determining the diameter of right knee joint at the end of day 28 as described in section D of this example, the rats in each group were sacrificed by virtue of 95% CO₂ asphyxiation. Next, a blood sample was collected from the heart of each rat through arterial puncture using a 25G needle, and was then subjected to centrifugation at 1,500 g and 25° C. for 10 minutes, so as to obtain a serum sample. Thereafter, the right knee joint tissue of the respective rat in each group was obtained from each rat carcass and was washed with 0.9% normal saline.

The resultant serum samples and right knee joint tissues were used for the following experiments.

F. Histopathologic Analysis

The respective right knee joint tissue obtained in section E of this example was subjected to a fixation treatment with a 10% neutral buffered formalin (BiOTnA Biotech, Cat. No. TABS06-4000) at room temperature for 24 hours. The fixed tissue sample was then embedded with paraffin, followed by slicing to obtain a tissue section having a thickness of 5 μm.

The tissue section was subjected to hematoxylin-eosin staining, and Safranin 0 and fast green staining using a staining protocol well-known to those skilled in the art, and was then observed under an optical microscope (Olympus, CKX41) at a magnification of 200×. Two areas of the respective tissue section were randomly selected and photographed, and pathological changes (such as cartilage damage, chondrocyte loss, and proteoglycan loss) in the respective tissue section was assessed according to the modified Mankin scoring system described in Pauli C. et al. (2012), Osteoarthritis Cartilage, 20:476-485. The degrees of cartilage damage, chondrocyte loss, and proteoglycan loss were scored on a scale from 1 to 5. The higher the scale, the higher the severity of OA is.

The data thus obtained were analyzed according to the method described in section 1 of the “General Procedures”.

Based on the result of the morphological observation (data not shown), it was found that in the pathological control group, the articular cartilage of the right knee joint had obvious cracks and an irregular surface, and severe chondrocyte and proteoglycan loss were observed, indicating that MIA successfully induced OA and cartilage lesions. In contrast, in the comparative group and the experimental groups 1 and 2, the articular cartilage of the right knee joint had fewer cracks and a smoother surface, and less chondrocyte and proteoglycan loss were observed.

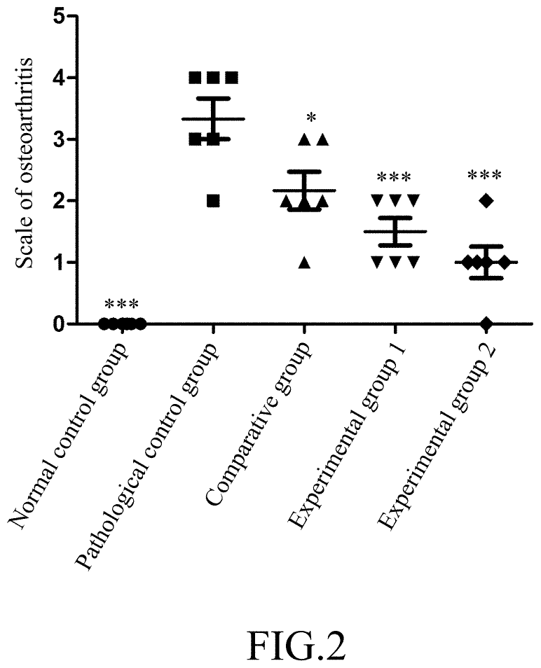

FIG. 2 shows the scoring result of OA. As shown in FIG. 2, the scales of OA determined in the experimental groups 1 and 2 were each significantly lower than that determined in the pathological control group.

These results indicate that EGF can exhibit satisfactory efficacy in alleviating OA and articular cartilage lesions, and this efficacy can be enhanced when EGF is used in combination with CS and HA.

G. Determination of OA Biomarkers and Inflammatory Markers in Serum Sample

The respective serum sample obtained in section E of this example was diluted 5-fold with RPMI 1640 medium. Thereafter, the content of cartilage oligomeric matrix protein (COMP) was determined using a rat COMP ELISA Kit (Cusabio, Cat. No. CSB-E13833r) in accordance with the manufacturer's instructions. In addition, the contents of TNF-α, IL-1β, IL-6, and IL-17A were respectively determined using a TNF-α ELISA Kit (Invitrogen, Cat. No. 88-7340-88), an IL-1β ELISA Kit (Invitrogen, Cat. No. 88-6010-22), an IL-6 ELISA Kit (Invitrogen, Cat. No. 88-50625-88), and an IL-17A ELISA Kit (Invitrogen, Cat. No. 88-7170-22) in accordance with the manufacturer's instructions. The content of prostaglandin E2 (PGE2) was determined using a PGE2 ELISA Kit (Cusabio, Cat. No. CSB-E07967r) in accordance with the manufacturer's instructions.

Moreover, the content of nitric oxide (NO) was determined using the Griess assay. Briefly, 100 μL of the respective serum sample obtained in section E of this example was mixed with 100 μL of a Griess reagent (containing 0.2% N-(1-Naphthyl)ethylenediamine, 2% sulfanilamide, and 5% phosphoric acid), followed by being left standing for reaction to proceed at room temperature for 10 minutes. The resultant reaction mixture was subsequently subjected to determination of absorbance at a wavelength of 540 nm (OD₅₄₀) using a spectrophotometer. The OD₅₄₀ values obtained were subsequently converted to concentration expressed in pg/mL based on a correlation curve previously prepared by plotting different known concentrations of sodium nitrate standards versus OD₅₄₀ values thereof.

The data thus obtained were analyzed according to the method described in section 1 of “General Procedures”.

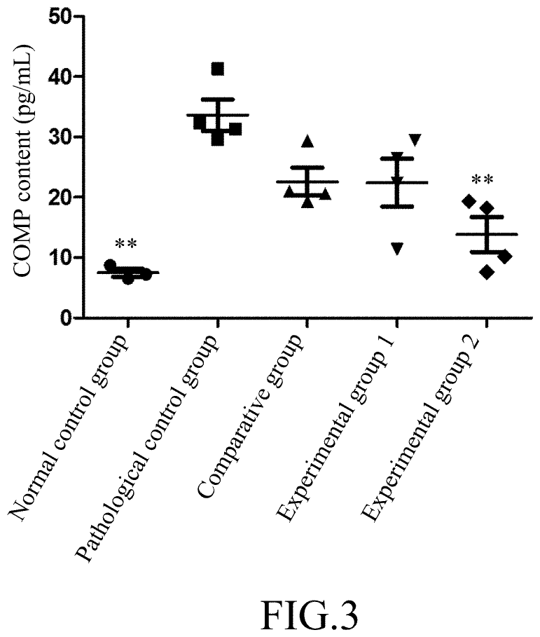

Referring to FIG. 3, the COMP contents determined in the comparative group and the experimental groups 1 and 2 were each lower than that determined in the pathological control group. In particular, the COMP content determined in the experimental group 2 was similar to that determined in the normal control group.

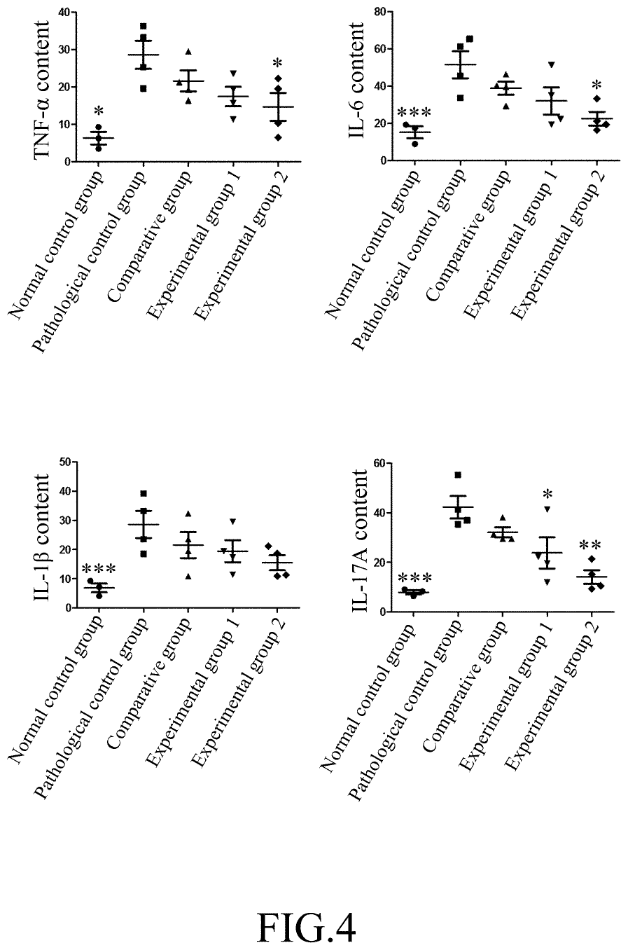

Referring to FIG. 4, the contents of TNF-α, IL-1β, IL-6, and IL-17A determined in the comparative group and the experimental groups 1 and 2 were each lower than those determined in the pathological control group.

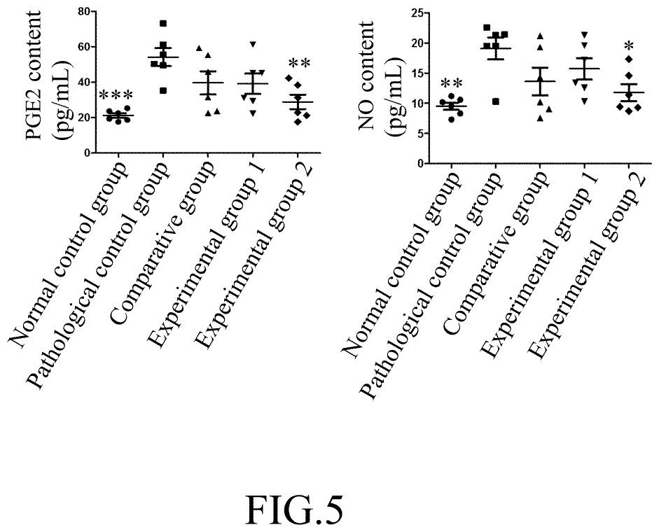

Referring to FIG. 5, the contents of PGE2 and NO determined in the comparative group and the experimental groups 1 and 2 were each lower than those determined in the pathological control group.

These results indicate that EGF can exhibit satisfactory efficacy in alleviating OA and joint inflammation symptoms, and this efficacy can be enhanced when EGF is used in combination with CS and HA.

### Example 2. Evaluation for the Effect of EGF in Alleviating Rheumatoid Arthritis (RA)

A. Experimental Mice

Female DBA/1 mice (8 weeks old, with a body weight of approximately 20 g) used in the following experiments were purchased from National Laboratory Animal Center, Taipei City, Taiwan. All the experimental mice were housed in an animal room under the following laboratory conditions: an alternating 12-hour light and 12-hour dark cycle, a temperature maintained at 21° C. to 23° C., and a relative humidity maintained at 45% to 60%. Furthermore, water and feed were provided ad libitum for all the experimental mice. All experimental procedures involving the experimental mice were in compliance with the legal provision of the Institutional Animal Care and Use Committee of National Chung Hsing University, Taiwan, and were carried out according to the Guide for the Care and Use of Laboratory Animals of National Institutes of Health (NIH).

B. Induction of RA and Administration of EGF

2 mg/mL of a Chick type II collagen (CII) solution (Chondrex Inc., Cat. No. 20012) (in 10 mM acetic acid) was mixed with complete Freund's adjuvant (Sigma Aldrich, Cat. No. F5881-10ML) at a ratio of 1:1 (v/v), so as to obtain a first CII emulsion. 2 mg/mL of a Chick type CII solution was mixed with incomplete Freund's adjuvant (ChemCruzx, Cat. No. sc-24648) at a ratio of 1:1 (v/v), so as to obtain a second CII emulsion.

The female DBA/1 mice were divided into five groups, including one normal control group, one pathological control group, one comparative group, and two experimental groups (i.e., experimental groups 1 to 2) (n=6 mice in each group). 200 μL of the first CII emulsion was injected subcutaneously into the tail of the respective mouse of the pathological control group, the comparative group, and the two experimental groups. The mice were subjected to a boost injection using 200 μL of the second CII emulsion on the 21^{st} day after the initial immunization, so as to induce the occurrence of RA. In addition, the rats of the normal control group were injected with an adjuvant solution (in PBS).

After completion of the boost injection, the respective mouse in the comparative group was intraperitoneally injected with 100 μL of 1% HA, the respective mouse in the experimental group 1 was intraperitoneally injected with 100 μL of the EGF solution (containing 20 μg/g EGF) prepared in section C of Example 1, the respective mouse in the experimental group 2 was intraperitoneally injected with 100 μL of the mixture solution (containing 20 μg/g EGF) prepared in section C of Example 1. Each mouse was administered once daily for a total period of 20 days.

In addition, the mice of the normal control group and pathological control group received no treatment.

C. Evaluation of RA Severity

At the end of the 21^{st} day after the initial immunization, the pathological symptoms (i.e., erythema and edema in the sole joint, ankle, and knee joint) in the respective mouse was assessed according to the visual arthritis scoring system described in Brand D. D. et al. (2007), Nat. Protoc., 2:1269-1275 and Milici A. J. et al. (2008), Arthritis Res. Ther., 10:R14. Thereafter, the abovementioned visual arthritis scoring system was performed every 3 days until the 42^{nd} day after the initial immunization (i.e., each mouse was scored 8 times). The degree of RA severity was scored on a scale from 1 to 8. The higher the scale, the higher the severity of RA is.

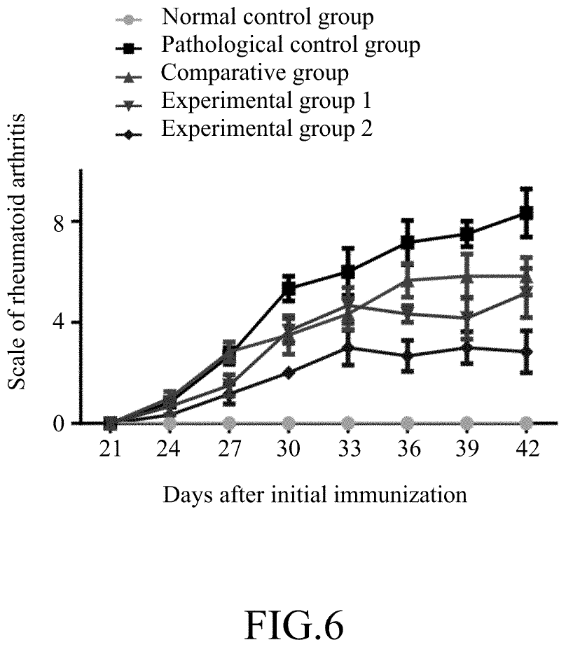

Referring to FIG. 6, the severity of RA in the pathological control group gradually increased over time. In comparison with the pathological control group, the severity of RA in the comparative group and the experimental group 1 increased slowly. In particular, the severity of RA in the experimental group 2 was lower than those in the pathological control group, the comparative group, and the experimental group 1, and a trend toward alleviation of RA was observed in the development of RA on the 33^{rd} to 42^{nd} days after the initial immunization.

These results indicate that EGF can exhibit satisfactory efficacy in alleviating RA and the symptoms associated with RA, and this efficacy can be enhanced when EGF is used in combination with CS and HA.

D. Histopathologic Analysis

After completion of the evaluation of RA severity as described in section C of this example, the mice in each group were sacrificed by virtue of 95% CO₂ asphyxiation, and the knee joint tissue of the hind leg and the sole tissue were obtained from each mouse carcass. The knee joint tissue of the hind leg of each mouse was subjected to histological sectioning, hematoxylin-eosin staining, and microscopic observation in sequence according to the methods described in section F of Example 1.

Next, the pathological symptoms (i.e., inflammatory cell infiltration, synovial hyperplasia, and cartilage surface erosion) in the respective tissue section was assessed according to the method described in Deng G. M. et al. (2005), Nat Med., 11:1066-1072. The degrees of inflammatory cell infiltration, synovial hyperplasia, and cartilage surface erosion were scored on a scale from 1 to 4. The higher the scale, the higher the severity of the pathological symptom is.

The data thus obtained were analyzed according to the method described in section 1 of the “General Procedures”.

Based on the result of the morphological observation (data not shown), it was found that in the comparative group and the experimental group 1, the cartilage structure was partially damaged, and a partially intact cartilage surface was observed. In particular, in the experimental group 2, the cartilage structure was complete and the cartilage surface was smooth, similar to the cartilage structure of the normal control group.

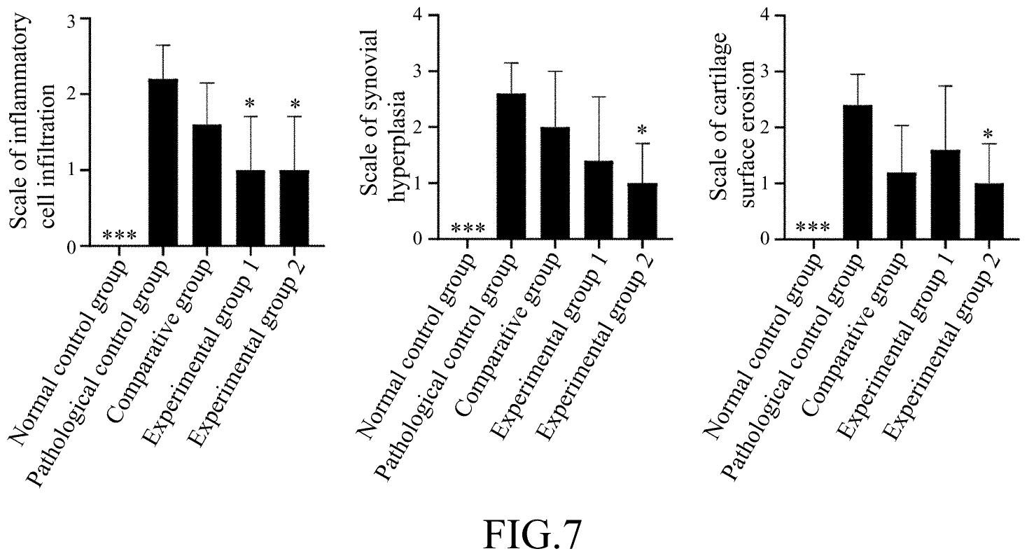

Referring to FIG. 7, the scales of the pathological symptoms (i.e., inflammatory cell infiltration, synovial hyperplasia, and cartilage surface erosion) determined in the experimental groups 1 and 2 were each apparently or significantly lower than those determined in the pathological control group.

These results indicate that EGF can exhibit satisfactory efficacy in alleviating RA and cartilage lesions associated with RA, and this efficacy can be enhanced when EGF is used in combination with CS and HA.

E. Determination of Inflammatory Markers in Sole Tissue

100 mg of the sole tissue of each mouse obtained in section D of this example was subjected to a freezing treatment with liquid nitrogen, followed by grinding. The resultant ground tissue was mixed with 1 mL of a tissue lysis buffer (Gold Biotechnology, Inc., Cat. No. GB-181-100) (containing protease inhibitor cocktail), followed by determining the contents of TNF-α, IL-1β, IL-6, IL-17A, IFN-γ, and IL-8 respectively using a TNF-α ELISA kit (BioLegend, Cat. No. 430904), an IL-1β ELISA kit (BioLegend, Cat. No. 432604), an IL-6 ELISA kit (BioLegend, Cat. No. 431304), an IL-17A ELISA kit (BioLegend, Cat. No. 432504), an IFN-γ ELISA kit (BioLegend, Cat. No. 430807), and an IL-8 ELISA kit (MyBioSource, Cat. No. MBS261967) in accordance with the manufacturer's instructions.

The data thus obtained were analyzed according to the method described in section 1 of “General Procedures”.

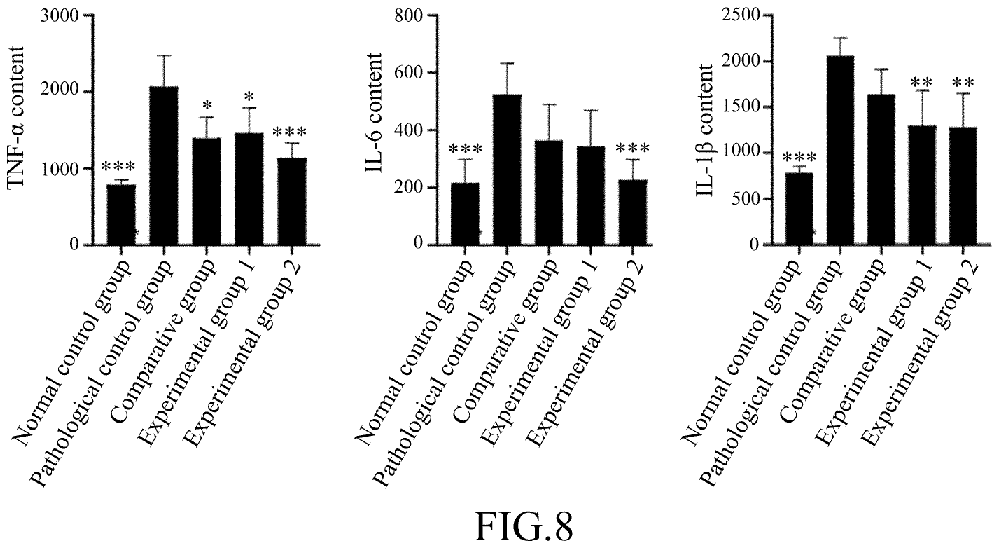

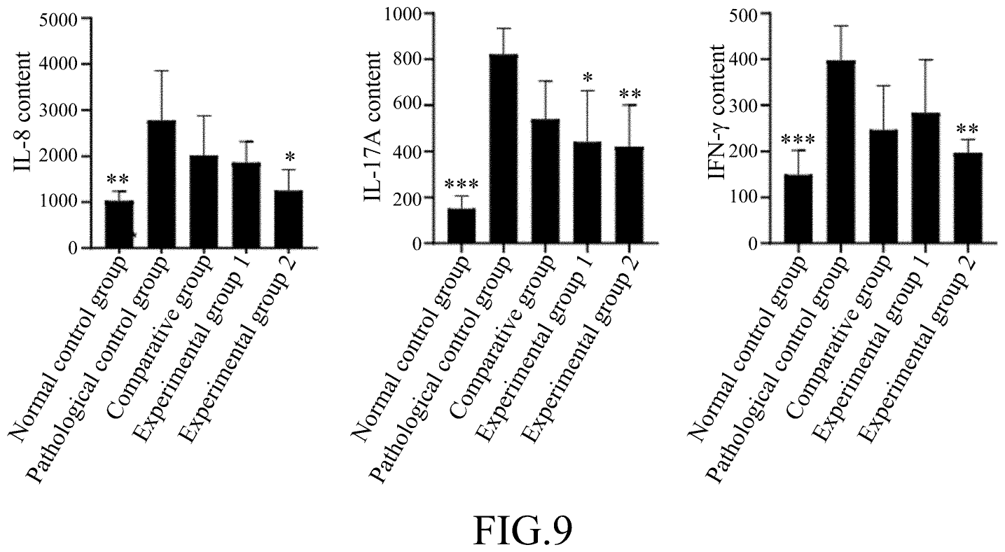

Referring to FIGS. 8 and 9, the contents of TNF-α, IL-1β, IL-6, IL-17A, IFN-γ, and IL-8 determined in the experimental groups 1 and 2 were each apparently or significantly lower than those determined in the pathological control group.

These results indicate that EGF can exhibit satisfactory efficacy in alleviating RA and inflammatory reactions caused by RA, and this efficacy can be enhanced when EGF is used in combination with CS and HA.

Summarizing the above test results, it is clear that EGF is capable of effectively alleviating arthritis (such as OA and RA), and may be further used in combination with a glycosaminoglycan (such as CS) and HA to exert desirable effects.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A method for alleviating arthritis, comprising administering to a subject in need thereof a composition containing epidermal growth factor.

2. The method as claimed in claim 1, wherein the arthritis is selected from the group consisting of osteoarthritis (OA), rheumatoid arthritis (RA), gouty arthritis (GA), psoriatic arthritis (PA), infectious arthritis (IA), and combinations thereof.

3. The method as claimed in claim 2, wherein the arthritis is osteoarthritis.

4. The method as claimed in claim 2, wherein the arthritis is rheumatoid arthritis.

5. The method as claimed in claim 1, wherein the composition further contains a glycosaminoglycan and hyaluronic acid.

6. The method as claimed in claim 5, wherein the epidermal growth factor, the glycosaminoglycan, and the hyaluronic acid are present in a weight ratio ranging from 1:10:100 to 1:1000:5000.

7. The method as claimed in claim 6, wherein the epidermal growth factor, the glycosaminoglycan, and the hyaluronic acid are present in a weight ratio of 1:100:1000.

8. The method as claimed in claim 5, wherein the glycosaminoglycan is selected from the group consisting of chondroitin sulfate, keratan sulfate, heparan sulfate, glucosamine sulfate, and combinations thereof.

9. The method as claimed in claim 8, wherein the glycosaminoglycan is chondroitin sulfate.

10. The method as claimed in claim 1, wherein the composition is formulated as a food product.

11. The method as claimed in claim 1, wherein the composition is formulated as a pharmaceutical composition.

12. The method as claimed in claim 11, wherein the pharmaceutical composition is in a dosage form selected from the group consisting of a parenteral dosage form, an oral dosage form, and a topical dosage form.
